(19) United States
(12) Patent Application Publication
(10) Pub. No.: US 2015/0182437 A1
(43) Pub. Date: Jul. 2, 2015

(54) COSMETIC COMPOSITION COMPRISING AN ANIONIC SURFACTANT, A SOLID FATTY ALCOHOL AND A SOLID FATTY ESTER, AND COSMETIC TREATMENT PROCESS

(71) Applicant: L'OREAL, Paris (FR)

(72) Inventors: Estelle Mathonneau, Paris (FR); Geraldine Fack, Levallois (FR); Laurent Chesneau, Levallois Perret (FR); Virginie Le Chaux, Franconville (FR)

(21) Appl. No.: 14/366,887

(22) PCT Filed: Dec. 19, 2012
(86) PCT No.: PCT/FR2012/052981

§ 371 Date: Sep. 25, 2014

Related U.S. Application Data
(60) Provisional application No. 61/582,733, filed on Jan. 3, 2012.
(60) Provisional application No. 61/582,741, filed on Jan. 3, 2012.

(30) Foreign Application Priority Data
Dec. 20, 2011 (FR) 1162037
Dec. 20, 2011 (FR) 1162038

(51) Int. Cl. A61K 8/46; A61K 8/34; A61K 8/37; A61Q 5/12

(52) U.S. Cl. CPC A61K 8/466; A61Q 5/12; A61K 8/463; A61K 8/342; A61K 8/37; A61K 2800/596

(57) ABSTRACT

The present invention relates to a cosmetic composition, in particular for conditioning the hair, comprising:—one or more sulphate, sulphonate or carboxylic anionic surfactants comprising a fatty chain,—one or more solid fatty alcohols, and—one or more solid fatty esters. The invention also relates to a process for cosmetic treatment, in particular for conditioning, of keratin materials, in particular sensitized hair, using said composition.

The present invention relates to a composition especially for conditioning keratin materials, in particular keratin fibers, comprising at least one particular anionic surfactant, at least one solid fatty alcohol and at least one solid fatty ester; the invention also relates to a cosmetic process for treating keratin materials, in particular keratin fibers, especially a process for conditioning said materials or fibers.

In the field of conditioners, use is generally made of conditioning agents, which may be a cationic surfactant, a cationic polymer, a silicone, an oil, a fatty substance, or a mixture thereof. These conditioning agents are used for improving the disentangling and softness of the hair, whether it is wet or dry hair, but may have a tendency to make the hair lank and dull.

As a result, the use of insoluble conditioning agents is greatly limited, firstly due to the stabilization difficulties of compositions comprising them, and secondly due to the cosmetic defects in terms of the lankness, charging and regreasing of keratin materials, associated with coarse or heterogeneous dispersions.

Insoluble conditioning agents, in particular fatty alcohols, are known and used in hair compositions, especially in documents JP2002-20791, JP9-30938, US 2009/005 449 and US 2009/005 460.

However, these compositions do not have high-quality cosmetic performance in terms of disentangling and smoothing, without making the head of hair lank or making it feel charged, and while maintaining an adequate level of working qualities.

The Applicant has now discovered that the use of compositions comprising at least one particular anionic surfactant, at least one solid fatty alcohol and at least one solid fatty ester makes it possible to obtain a composition with very good cosmetic properties: in particular, the sleeking, softness, suppleness and sheen are improved, especially on sensitized hair. In the case of frizzy and/or curly hair, a decrease in volume is also observed, enabling better control of the head of hair.

It has also been found that the compositions according to the invention also afford color protection on washing artificially dyed hair.

One subject of the invention is thus a non-coloring and non-oxidizing cosmetic composition comprising one or more sulfate, sulfonate or carboxylic anionic surfactants, one or more solid fatty alcohols and one or more solid fatty esters, said anionic surfactant(s) being such that at least 50% by weight of them comprise fatty chains containing a number of carbon atoms greater than or equal to 14.

Another subject of the invention is a cosmetic treatment process for keratin materials, in particular human keratin fibers and most especially the hair, or even sensitized hair, using the composition according to the invention.

It is most particularly a process for conditioning said keratin materials.

In the present description, the term “at least one” is equivalent to the expression “one or more”.

According to the present invention, the term “non-coloring composition” means a composition not containing any dye for keratin fibers such as direct dyes or oxidation dye precursors (bases and couplers). If they are present, their content does not exceed 0.005% by weight relative to the total weight of the composition. Specifically, at such a content, only the composition would be dyed, i.e. no dyeing effect would be observed on the keratin fibers.

According to the present invention, the term “non-oxidizing composition” means a composition not containing any oxidizing agent usually used in keratin fiber treatments, such as hydrogen peroxide, urea peroxide, alkali metal bromates or ferri-cyanides, peroxygenated salts, for instance alkali metal or alkaline-earth metal persulfates, perborates, peracids and precursors thereof, and percarbonates of alkali metals or alkaline-earth metals, and peracids and precursors thereof. If they are present, their content does not exceed 0.005% by weight relative to the total weight of the composition.

In the present description, a species is termed as being “anionic” when it bears at least one permanent negative charge or when it can be ionized as a negatively charged species, under the conditions of use of the compositions of the invention (for example the medium or the pH) and not comprising any cationic filler.

### Anionic Surfactants

The sulfate or sulfonate anionic surfactants according to the invention are anionic surfactants comprising at least one sulfate (—OSO₃H or —OSO₃⁻) function and/or one sulfonate (—SO₃H or —SO₃⁻) function.

The carboxylic anionic surfactants according to the invention are anionic surfactants comprising at least one carboxylic function —COOH or —COO⁻.

It is recalled that said sulfate, sulfonate or carboxylic anionic surfactants are such that at least 50% by weight of them comprise fatty chains containing a number of carbon atoms greater than or equal to 14.

The sulfate or sulfonate anionic surfactants that may be used in the composition according to the invention are especially chosen from salts, in particular alkali metal salts such as the sodium salts, the ammonium salts, the amine salts, the amino alcohol salts or the alkaline-earth metal salts, for example the magnesium salts, of the following types: alkyl sulfates, alkyl ether sulfates, alkylamido ether sulfates, alkylaryl polyether sulfates, monoglyceride sulfates; alkylsulfonates, alkylamide-sulfonates, alkylarylsulfonates, α-olefin sulfonates, paraffin sulfonates; alkyl sulfosuccinates, alkyl ether sulfosuccinates, alkylamide sulfosuccinates; alkyl sulfoacetates, the alkyl and acyl groups of all these compounds preferably comprising from 14 to 30 carbon atoms and better still from 16 to 22 carbon atoms and the aryl group preferably denoting a phenyl or benzyl group.

Preferably, the sulfate or sulfonate anionic surfactants are chosen from C16-C18 alkyl sulfates and C16-18 alkyl ether sulfates, and mixtures thereof, in particular in the form of salts of alkali metals or alkaline-earth metals, with ammonium, with amine or with amino alcohol.

The carboxylic anionic surfactants that may be used in the composition according to the invention are especially chosen from salts, in particular the alkali metal salts such as the sodium salts, the ammonium salts, the amine salts, the amino alcohol salts or the alkaline-earth metal salts, for example the magnesium salts, of the following types; alkyl sulfosuccinates, alkyl ether sulfosuccinates, alkylamide sulfosuccinates; acyl glycinates, acylsarcosinates and acylglutamates, the alkyl and acyl groups of all these compounds comprising from 14 to 30 carbon atoms and better still from 16 to 22 carbon atoms.

Use may also be made of alkyl monoesters of polyglycoside-polycarboxylic acids such as alkyl glucoside citrates, alkyl polyglycoside tartrates and alkyl polyglycoside sulfosuccinates, and alkylsulfosuccinamates, the alkyl or acyl group of all these compounds comprising from 14 to 30 carbon atoms and better still from 16 to 22 carbon atoms.

Mention may also be made of acyllactylates, the acyl group of which comprises from 14 to 30 carbon atoms and better still from 16 to 22 carbon atoms.

Mention may also be made of alkyl-D-galactosideuronic acids and salts thereof and also polyoxyalkylenated (C₁₄₋₃₀)alkyl ether carboxylic acids, polyoxyalkylenated (C₁₄₋₃₀)alkyl(C₆₋₃₀)aryl ether carboxylic acids, polyoxyalkylenated (C₁₄₋₃₀)alkylamido ether carboxylic acids and salts thereof, in particular those comprising from 2 to 50 ethylene oxide units, and mixtures thereof.

Preferably, the carboxylic anionic surfactants according to the invention do not comprise any sulfate or sulfonate functions.

Preferably, the carboxylic anionic surfactants according to the invention are chosen from acylglutamates such as stearoyl glutamates, acyl sarcosinates such as palmitoyl sarcosinates, acyl lactylates such as behenoyl lactylates, and mixtures thereof, in particular in the form of alkali metal or alkaline-earth metal, ammonium, amine or amino alcohol salts. Mention may be made more particularly of disodium stearoyl glutamate, sodium palmitoyl sarcosinate and sodium behenoyl lactylate.

The anionic surfactant(s) according to the invention are preferably present in the composition in a total amount ranging from 0.1% to 20% by weight, preferentially from 0.5% to 10% by weight and better still from 1% to 5% by weight relative to the total weight of the composition.

### Solid Fatty Alcohols

The “solid fatty alcohols” are solid at room temperature (25° C.) and at atmospheric pressure (780 mmHg or 1 atm.); they are water-insoluble, i.e. they have a solubility in water of less than 1% by weight and preferably less than 0.5% by weight.

The term “fatty alcohol” means a long-chain aliphatic alcohol comprising from 8 to 40 carbon atoms, preferably from 12 to 34 or even from 12 to 30 carbon atoms, and comprising at least one hydroxyl group OH. These fatty alcohols are neither oxyalkylenated nor glycerolated.

Preferably, the solid fatty alcohols are of structure R—OH with R denoting a linear alkyl group, optionally substituted with one or more hydroxyl groups, comprising from 12 to 40, better still from 12 to 34, or even from 12 to 30 and most preferentially from 12 to 24 carbon atoms.

The solid fatty alcohols that that may be used in the context of the invention, are more particularly chosen from:
lauryl alcohol (1-dodecanol);
myristyl alcohol (1-tetradecanol);
cetyl alcohol (1-hexadecanol);
stearyl alcohol (1-octadecanol);
arachidyl alcohol (1-eicosanol);
behenyl alcohol (1-docosanol);
lignoceryl alcohol (1-tetracosanol);
ceryl alcohol (1-hexacosanol);
montanyl alcohol (1-octacosanol);
myricyl alcohol (1-triacontanol);

and mixtures thereof.

More particularly, the solid fatty alcohol is chosen from cetyl alcohol, stearyl alcohol, behenyl alcohol and mixtures thereof such as cetylstearyl alcohol or cetearyl alcohol.

The fatty alcohols may be mixtures, which means, for example, that several species may coexist in a commercial product, especially of different chain lengths, in the form of a mixture.

The solid fatty alcohol(s) according to the invention are preferably present in the composition in an amount of from 0.01% to 30% by weight, especially from 0.1% to 15% by weight, preferentially from 0.5% to 12% by weight and better still from 2% to 12% by weight, or even from 4% to 10% by weight, relative to the total weight of the composition.

Preferably, the weight ratio of the solid fatty alcohol(s)/anionic surfactant(s) according to the invention ranges from 1 to 15 and better still from 2 to 10.

### Solid Fatty Esters

The composition of the invention also comprises one or more fatty esters that are solid at room temperature (25° C.) and at atmospheric pressure (1 atm.).

Preferably, the solid fatty esters are esters of monoalcohols, especially of monoalcohols comprising at least 10 carbon atoms, and better still of saturated monoalcohols comprising at least 10 carbon atoms.

Preferentially, the solid fatty esters are esters of saturated carboxylic acids comprising at least 10 carbon atoms and of saturated monoalcohols comprising at least 10 carbon atoms.

The saturated carboxylic acids and/or monoalcohols may be linear or branched.

The saturated carboxylic acids preferably comprise from 10 to 30 carbon atoms and more particularly from 12 to 24 carbon atoms. They may optionally be hydroxylated.

The saturated monoalcohols preferably comprise from 10 to 30 carbon atoms and more particularly from 12 to 24 carbon atoms.

Preferably, the solid fatty esters are chosen from myristyl myristate, cetyl myristate, stearyl myristate, myristyl palmitate, cetyl palmitate, stearyl palmitate, myristyl stearate, cetyl stearate and stearyl stearate, and also mixtures thereof.

Said solid fatty ester(s) are preferably present in the composition in an amount of between 0.1% and 10% by weight and preferably between 0.5% and 5% by weight relative to the total weight of the composition.

### Liquid Fatty Substances

The composition according to the invention may also comprise one or more liquid fatty substances.

The term “fatty substance” means an organic compound that is insoluble in water at room temperature (25° C.) and at atmospheric pressure (760 mmHg), i.e. whose solubility is less than 5%, preferably 1% and even more preferentially 0.1%.

They generally have in their structure at least one hydrocarbon-based chain comprising at least 6 carbon atoms and/or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane. These fatty substances are neither polyoxyethylenated nor polyglycerolated.

The fatty substances according to the invention are liquid at room temperature (25° C.) and at atmospheric pressure (780 mmHg).

They are preferably chosen from liquid fatty alcohols, liquid fatty esters, silicone oils, C₆-C₁₆ hydrocarbons, hydrocarbons containing more than 16 carbon atoms, non-silicone oils of animal origin, plant oils of triglyceride type, synthetic triglycerides and fluoro oils, and mixtures thereof.

The liquid fatty alcohols, in particular the C₁₀-C₃₄ alcohols, have branched carbon-based chains or contain one or more (preferably 1 to 3) unsaturations.

They are preferably branched and/or unsaturated, and comprise from 12 to 40 carbon atoms. They are non-oxyalkylenated and non-glycerolated.

The liquid fatty alcohols preferably have the structure R—OH, in which R denotes a branched C₁₂-C₂₄ alkyl or C₁₂-C₂₄ alkenyl group, R possibly being substituted with one or more hydroxyl groups. Preferably, R does not contain any hydroxyl groups. Preferably, the liquid fatty alcohol is a branched saturated alcohol.

Examples that may be mentioned include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol and 2-tetradecyl-1-cetanol, and mixtures thereof. Preferentially, the liquid fatty alcohol is 2-octyl-1-dodecanol.

The liquid fatty alcohols may be mixtures, which means that several species may coexist in a commercial product, especially of different chain lengths, in the form of a mixture.

The liquid fatty esters that may be used may be esters of monoalcohols or of polyols with monoacids or polyacids, at least one of the alcohols and/or acids comprising at least one chain of more than 7 carbon atoms. Preferably, the liquid fatty ester is chosen from fatty acid esters of monoalcohols. Preferably, at least one of the alcohols and/or acids is branched.

Examples that may be mentioned include isopropyl myristate, isononyl palmitate, isopropyl isononanoate, 2-ethylhexyl palmitate, 2-hexyldecyl laurate, 2-octyldecyl palmitate and 2-octyldodecyl myristate, and mixtures thereof.

The silicones that can be used in accordance with the invention are in the form of oils.

Preferably, the silicone is chosen from polydialkylsiloxanes, especially polydimethylsiloxanes (PDMS), and organomodified polysiloxanes comprising at least one functional group chosen from amino groups and alkoxy groups. Organopolysiloxanes are defined in greater detail in Walter Noll's Chemistry and Technology of Silicones (1968), Academic Press. They may be volatile or nonvolatile.

When they are volatile, the silicones are more particularly chosen from those with a boiling point of between 60° C. and 260° C., and even more particularly from:

(i) cyclic polydialkylsiloxanes comprising from 3 to 7 and preferably from 4 to 5 silicon atoms. These are, for example, octamethylcyclotetrasiloxane sold in particular under the name Volatile Silicone® 7207 by Union Carbide or Silbione® 70045 V 2 by Rhodia, decamethylcyclopentasiloxane sold under the name Volatile Silicone® 7158 by Union Carbide, and Silbione® 70045 V 5 by Rhodia, and mixtures thereof.

Mention may also be made of cyclocopolymers of the dimethylsiloxane/methylalkylsiloxane type, such as Volatile Silicone® FZ 3109 sold by the company Union Carbide, of formula:

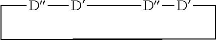

where D″:

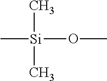

where D′:

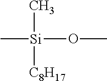

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones falling within this category are also described in the article published in Cosmetics and Toiletries, Vol. 91, January 76, pp. 27-32, Todd & Byers, Volatile Silicone Fluids for Cosmetics.

Use is preferably made of nonvolatile polydialkylsiloxanes, polyorganosiloxanes modified with the organofunctional groups above, and mixtures thereof.

These silicones are more particularly chosen from polydialkylsiloxanes, among which mention may be made mainly of polydimethylsiloxanes having trimethylsilyl end groups. The viscosity of the silicones is measured at 25° C. according to ASTM Standard 445 Appendix C.

Mention may be made, among these polydialkylsiloxanes, without limitation, of the following commercial products: the Silbione® oils of the 47 and 70 047 series or the Mirasil® oils sold by Rhodia, for instance the 70 047 V 500 000 oil; the oils of the Mirasil® series sold by the company Rhodia; the oils of the 200 series from the company Dow Corning, such as DC200 with a viscosity of 60 000 mm²/s; the Viscasil® oils from General Electric and certain oils of the SF series (SF 96, SF 18) from General Electric.

Mention may also be made of polydimethylsiloxanes bearing dimethylsilanol end groups known under the name dimethiconol (CTFA), such as the oils of the 48 series from the company Rhodia.

In this category of polydialkylsiloxanes, mention may also be made of the products sold under the names Abil Wax® 9800 and 9801 by the company Goldschmidt, which are poly(C₁-C₂₀)dialkylsiloxanes.

Products that may more particularly be used in accordance with the invention are mixtures such as mixtures formed from a polydimethylsiloxane hydroxylated at the chain end, or dimethiconol (CTFA), and from a cyclic polydimethylsiloxane, also known as cyclomethicone (CTFA), such as the product Q2-1401 sold by the company Dow Corning.

The organomodified silicones that may be used in accordance with the invention are silicones as defined previously and comprising in their structure one or more organofunctional groups attached via a hydrocarbon-based group.

Besides the silicones described above, the organomodified silicones may be polydiarylsiloxanes, especially polydiphenylsiloxanes, and polyalkylarylsiloxanes functionalized with the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names: the Silbione® oils of the 70 641 series from Rhodia; the oils of the Rhodorsil® 70 633 and 763 series from Rhodia; the oil Dow Corning 556 Cosmetic Grade Fluid from Dow Corning; the silicones of the PK series from Bayer, such as the product PK20; the silicones of the PN and PH series from Bayer, such as the products PN1000 and PH1000; certain oils of the SF series from General Electric, such as SF 1023, SF 1154, SF 1250 and SF 1265.

Among the organomodified silicones, mention may be made of polyorganosiloxanes comprising: (i) substituted or unsubstituted amino groups, such as the products sold under the name GP 4 Silicone Fluid and GP 7100 by the company Genesee, the substituted amino groups in particular being C1-C4 aminoalkyl groups; (ii) alkoxylated groups, such as the product sold under the name Silicone Copolymer F-755 by SWS Silicones.

In one variant of the invention, the silicones are not organomodified.

The C6-C16 hydrocarbons are preferably linear or branched, and optionally cyclic, and are preferably alkanes. Mention may be made of hexane, dodecane or isoparaffins, such as isohexadecane or isodecane.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, pracaxi oil, argan oil, castor oil, avocado oil, caprylic/capric acid triglycerides, for instance those sold by the company Stéarineries Dubois or those sold under the names Miglyol® 810, 812 and 818 by the company Dynamit Nobel, jojoba oil and shea butter oil.

The linear or branched hydrocarbons of mineral or synthetic origin containing more than 16 carbon atoms are preferably chosen from liquid paraffins, petroleum jelly, liquid petroleum jelly, polydecenes and hydrogenated polyisobutene such as Parleam®.

The fluoro oils may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

Preferably, the liquid fatty substances are chosen from silicone oils and liquid fatty alcohols, and mixtures thereof. Preferentially, the liquid fatty substances are chosen from silicone oils.

When they are present, the liquid fatty substances are preferably present in an amount ranging from 0.01% to 20% by weight, preferentially from 0.1% to 10% by weight and better still from 0.5% to 5% by weight, relative to the total weight of the composition.

### Other Ingredients

The cosmetic composition may also comprise at least one nonionic surfactant and/or at least one amphoteric surfactant.

The nonionic surfactants that may be used are compounds that are well known; (see especially in this regard the Handbook of Surfactants by M. R. Porter, published by Blackie & Son (Glasgow and London), 1991, pp. 116-178. They are especially chosen from alcohols, α-diols and (C₁₋₂₀)alkylphenols, these compounds being polyethoxylated, polypropoxylated or polyglycerolated and bearing a fatty chain comprising, for example, from 8 to 30 and preferably from 8 to 22 carbon atoms, the number of ethylene oxide or propylene oxide groups possibly ranging especially from 2 to 50, and the number of glycerol groups possibly ranging especially from 2 to 30.

Mention may also be made of condensates of ethylene oxide and of propylene oxide with fatty alcohols; polyethoxylated fatty amides preferably bearing from 2 to 30 ethylene oxide units, polyglycerolated fatty amides containing on average 1 to 5 and in particular 1.5 to 4 glycerol groups, polyethoxylated fatty acid esters of sorbitan preferably containing from 2 to 40 ethylene oxide units, fatty acid esters of sucrose, fatty acid esters of polyethylene glycol, (C₆₋₂₄ alkyl)polyglycosides, N-(C₆₋₂₄ alkyl)glucamine derivatives, amine oxides such as (C₁₀C₁₄alkyl)amine oxides or N(C₁₀₋₁₄ acyl)aminopropylmorpholine oxides.

Among the nonionic surfactants, use is preferably made of (C₆₋₂₄ alkyl)polyglycosides, and more particularly (C₈₋₁₈ alkyl)polyglycosides, polyethoxylated fatty acid esters of sorbitan and polyethoxylated fatty alcohols.

The amphoteric surfactants that may be used in the present invention may especially be secondary or tertiary aliphatic amine derivatives in which the aliphatic group is a linear or branched chain containing from 8 to 22 carbon atoms and containing at least one anionic group, for instance a carboxylate, sulfonate, sulfate, phosphate or phosphonate group. Mention may be made in particular of (C₈₋₂₀alkyl)betaines, sulfobetaines, (C₈₋₂₀ alkyl)amido(C₂₋₈ alkyl)betaines and (C₈₋₂₀ alkyl)amido(C₂₋₈ alkyl)sulfobetaines.

Among the amine derivatives, mention may be made of the products sold under the name Miranol®, as described in U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures (1) and (2):

Rₐ—CONHCH₂CH₂—N(R_{b})(R_{c})(CH₂COO⁻) (1)

in which:

Rₐ represents an alkyl group derived from an acid Rₐ—COOH present in hydrolyzed coconut oil, or a heptyl, nonyl or undecyl group,

R_{b} represents a β-hydroxyethyl group, and

R_{c} represents a carboxymethyl group;

and

Rₐ′—CONHCH₂CH₂—N(B)(C) (2)

in which:

B represents —CH₂CH₂OX′,

C represents —(CH₂)₂—Y′, with z=1 or 2,

X′ represents the —CH₂CH₂—COOH group or a hydrogen atom,

Y′ represents —COOH or the —CH₂—CHOH—SO₃H group,

Rₐ′ represents an alkyl group of an acid Rₐ′—COOH present in copra oil or in hydrolyzed linseed oil, a saturated or unsaturated C₇-C₂₃ alkyl group, especially a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

By way of example, mention may be made of the cocoamphodiacetate sold by the company Rhodia under the trade name Miranol® C2M Concentrate.

Among the amphoteric surfactants mentioned above that are preferably used are (C₈₋₂₀ alkyl)betaines, (C₈₋₂₀ alkyl)amido(C₂₋₈ alkyl)betaines and alkylamphodiacetates, and mixtures thereof.

The nonionic and/or amphoteric surfactants are preferably present in the composition according to the invention in an amount ranging from 0.1% to 10% by weight and better still from 0.5% to 5% by weight relative to the total weight of the composition.

Preferably, the total amount of surfactants in the composition according to the invention ranges from 1% to 20% by weight, better still from 1% to 10% by weight and even better still from 1% to 5% by weight relative to the total weight of the composition.

The composition according to the invention may also comprise one or more cationic polymers. The cationic polymers that may be used in accordance with the present invention may be chosen from any of those already known per se as improving the cosmetic properties of hair, namely, especially, those described in patent application EP-A-0 337 354 and in French patent applications FR-A-2 270 846, 2 383 660, 2 598 611, 2 470 596 and 2 519 863.

Even more generally, for the purposes of the present invention, the term “cationic polymer” denotes any polymer comprising cationic groups and/or groups that can be ionized into cationic groups.

The preferred cationic polymers are chosen from those that contain units comprising primary, secondary, tertiary and/or quaternary amine groups that may either form part of the main polymer chain or may be borne by a side substituent directly connected thereto.

The cationic polymers used generally have a number-average or weight-average molar mass of between 500 and 5×10⁶ approximately and preferably between 10³ and 3×10⁶ approximately.

Among the cationic polymers that may be mentioned more particularly are polymers of the polyamine, polyaminoamide and polyquaternary ammonium type. These are known products.

The polymers of polyamine, polyamidoamide and polyquaternary ammonium type that can be used in accordance with the present invention, and that can in particular be mentioned, are those described in French patents No. 2 505 348 or 2 542 997.

Among these polymers, mention may be made of:
(1) homopolymers or copolymers derived from acrylic or methacrylic esters or amides and comprising at least one of the units of the following formulae:

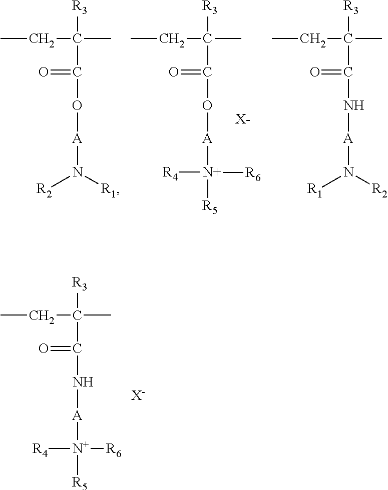

in which:

R3, which may be identical or different, denote a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R4, R5 and R6, which may be identical or different, represent an alkyl group containing from 1 to 18 carbon atoms or a benzyl radical and preferably an alkyl group containing from 1 to 6 carbon atoms;

R1 and R2, which may be identical or different, represent hydrogen or an alkyl group containing from 1 to 6 carbon atoms, and preferably methyl or ethyl;

X denotes an anion derived from a mineral or organic acid, such as a methosulfate anion or a halide such as chloride or bromide.

The copolymers of family (1) may also contain one or more units derived from comonomers that may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyl-lactams such as vinylpyrrolidone or vinylcaprolactam, and vinyl esters.

Accordingly, these copolymers of class (1) may include the following:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, described, for example, in patent application EP-A-080 976 and sold under the name Bina Quat P 100 by the company Ciba Geigy,
the copolymer of acrylamide and of methacryloyloxyethyltrimethylammonium methosulfate sold under the name Reten by the company Hercules,
quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers sold in particular under the name Styleze CC 10 by ISP,
quaternized vinylpyrrolidone/dimethylaminopropylmethacrylamide copolymers such as the product sold under the name Gafquat HS 100 by the company ISP,
preferably crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of said copolymer in mineral oil may be used more particularly. This dispersion is sold under the name Salcare® SC 92 by the company Ciba. A crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Ciba.
(2) cationic polysaccharides, especially cationic celluloses and cationic galactomannan gums. Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.
(3) the cellulose ether derivatives containing quaternary ammonium groups, described in French patent 1 492 597, and in particular the polymers sold under the names Ucare Polymer “JR” (JR 400T, JR 125 and JR 30M) or “LR” (LR 400 or LR 30M) by the company Amerchol. These polymers are also defined in the CTFA dictionary as quaternary ammoniums of hydroxyethyl cellulose that have reacted with an epoxide substituted with a trimethylammonium group.
(4) cationic cellulose copolymers or cellulose derivatives grafted with a water-soluble quaternary ammonium monomer are described especially in U.S. Pat. No. 4,131,576, such as hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The commercial products corresponding to this definition are more particularly the products sold under the names Celquat L 200 and Celquat H 100 by the company National Starch.
(5) the cationic galactomannan gums described more particularly in U.S. Pat. Nos. 3,589,578 and 4,031,307, in particular guar gums comprising cationic trialkylammonium groups. Guar gums modified with a 2,3-epoxypropyltrimethylammonium salt (e.g. chloride) are used, for example.

Such products are sold in particular under the trade names Jaguar C13 S, Jaguar C 15, Jaguar C 17 or Jaguar C162 by the company Rhodia.
(6) polymers formed from piperazinyl units and divalent alkylene or hydroxyalkylene radicals bearing straight or branched chains, optionally interrupted with oxygen, sulfur or nitrogen atoms or with aromatic or heterocyclic rings, and also the oxidation and/or quaternization products of these polymers. Such polymers are described, in particular, in French patents 2 162 025 and 2 280 361.
(7) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bishaloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are especially described in French patents 2 252 840 and 2 368 508.
(8) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Such polymers are especially described in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.
(9) polymers obtained by reaction of a polyalkylene polyamine containing two primary amine groups and at least one secondary amine group with a dicarboxylic acid chosen from diglycolic acid and saturated aliphatic dicarboxylic acids containing from 3 to 8 carbon atoms. The mole ratio between the polyalkylene polyamine and the dicarboxylic acid is between 0.8:1 and 1.4:1; the polyamino amide resulting therefrom is reacted with epichlorohydrin in a mole ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.
(10) alkyldiallylamine or dialkyldiallylammonium cyclopolymers, such as homopolymers or copolymers comprising, as the main chain constituent, units corresponding to formula (I) or (II):

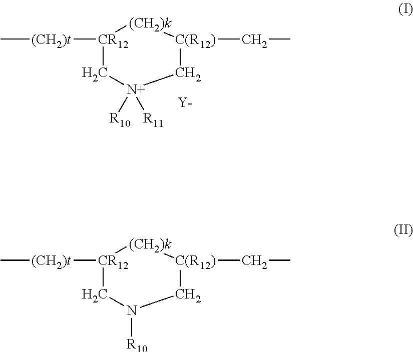

in which k and t are equal to 0 or 1, the sum k+t being equal to 1; R12 denotes a hydrogen atom or a methyl radical; R10 and R11, independently of each other, denote an alkyl group having from 1 to 6 carbon atoms, a hydroxyalkyl group in which the alkyl group has preferably 1 to 5 carbon atoms, a lower (C₁-C₄) amidoalkyl group, or R10 and R11 may denote, jointly with the nitrogen atom to which they are attached, heterocyclic groups, such as piperidinyl or morpholinyl; Y⁻ is an anion such as bromide, chloride, acetate, borate, citrate, tartrate, bisulfate, bisulfite, sulfate or phosphate. These polymers are especially described in French patent 2 080 759 and in its certificate of addition 2 190 406; R10 and R11, independently of each other, preferably denote an alkyl group containing from 1 to 4 carbon atoms.

Among the polymers defined above, mention may be made more particularly of the dimethyldiallylammonium salt (for example chloride) homopolymer, for example sold under the name Merquat 100 by the company Nalco (and homologs thereof of low weight-average molar masses) and the copolymers of diallyldimethylammonium salts (for example chloride) and of acrylamide, sold especially under the names Merquat 550 and Merquat 7SPR.
(11) diquaternary ammonium polymers comprising repeating units corresponding to the formula:

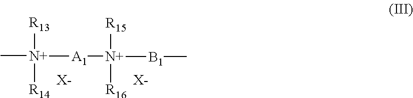

in which formula (III):

R13, R14, R15 and R16, which may be identical or different, represent aliphatic, alicyclic or arylaliphatic radicals comprising from 1 to 20 carbon atoms, or lower hydroxyalkylaliphatic radicals, or else R13, R14, R15 and R16, together or separately, constitute, with the nitrogen atoms to which they are attached, heterocycles optionally comprising a second non-nitrogen heteroatom, or else R13, R14, R15 and R16 represent a linear or branched C₁-C₆ alkyl radical substituted with a nitrile, ester, acyl, amide or —CO—O—R17-D or —CO—NH—R17-D group in which R17 is an alkylene and D is a quaternary ammonium group;

A1 and B1 represent polymethylene groups comprising from 2 to 20 carbon atoms, which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A1, R13 and R15 may form, with the two nitrogen atoms to which they are attached, a piperazine ring; moreover, if Al denotes a saturated or unsaturated, linear or branched alkylene or hydroxyalkylene radical, B1 may also denote a group (CH₂)ₙ—CO-D-OC—(CH₂)ₙ—

in which D denotes:

a glycol residue of formula —O—Z—O—, in which Z denotes a linear or branched hydrocarbon-based radical, or a group corresponding to one of the following formulae:

—(CH₂—CH₂—O)ₓ—CH₂—CH₂— or —[CH₂—CH(CH₃)—O]_{y}—CH₂—CH(CH₃)—

in which x and y denote an integer from 1 to 4, representing a defined and unique degree of polymerization or any number from 1 to 4 representing an average degree of polymerization;

a bis-secondary diamine residue such as a piperazine derivative;

a bis-primary diamine residue of formula: —NH—Y—NH—, where Y denotes a linear or branched hydrocarbon-based radical, or alternatively the divalent radical —CH₂—CH₂—S—S—CH₂—CH₂—;

a ureylene group of formula: —NH—CO—NH—;

preferably, X⁻ is an anion such as chloride or bromide.

These polymers have a number-average molar mass generally of between 1000 and 100 000.

Polymers of this type are described especially in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may be made more particularly of polymers that are composed of repeating units corresponding to the formula:

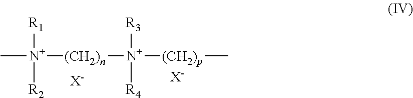

in which R1, R2, R3 and R4, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A particularly preferred compound of formula (IV) is that for which R1, R2, R3 and R4 represent a methyl radical and n=3, p=6 and X=Cl, known as Hexadimethrine chloride according to the INCI (CTFA) nomenclature.
(12) polyquaternary ammonium polymers comprising units of formula (V):

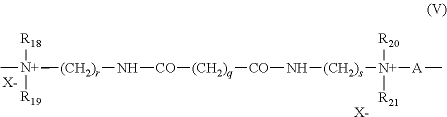

in which:

R18, R19, R20 and R21, which may be identical or different, represent a hydrogen atom or a methyl, ethyl, propyl, β-hydroxyethyl, β-hydroxypropyl or —CH₂CH₂(OCH₂CH₂)ₚOH radical,

where p is equal to 0 or to an integer between 1 and 6, with the proviso that R18, R19, R20 and R21 do not simultaneously represent a hydrogen atom,

r and s, which may be identical or different, are integers between 1 and 6,

q is equal to 0 or to an integer between 1 and 34,

X⁻ denotes an anion such as a halide,

A denotes a radical of a dihalide or, preferably, —CH₂—CH₂—O—CH₂—CH₂—.

Such compounds are described especially in patent application EP-A-122 324.

Among these, mention may be made, for example, of the products Mirapol® A 15, Mirapol® AD1, Mirapol® AZ1 and Mirapol® 175, sold by the company Miranol.
(13) quaternary polymers of vinylpyrrolidone and of vinylimidazole, for instance the products sold under the names Luviquat® FC 905, FC 550 and FC 370 by the company BASF.
(14) polyamines such as Polyquart® H sold by Cognis, referred to under the name Polyethylene glycol (15) tallow polyamine in the CTFA dictionary.

Other cationic polymers that may be used in the context of the invention are cationic proteins or cationic protein hydrolyzates, polyalkyleneimines, in particular polyethyleneimines, polymers comprising vinylpyridine or vinylpyridinium units, condensates of polyamines and of epichlorohydrin, quaternary polyureylenes and chitin derivatives.

Among the cationic polymers mentioned above, the ones that may preferably be used are quaternary cellulose ether derivatives such as the products sold under the name JR 400 by the company Amerchol, cationic cyclopolymers, in particular dimethyldiallylammonium salt (for example chloride) homopolymers or copolymers, products sold under the names Merquat 100, Merquat 550 and Merquat S by the company Nalco, and homologs thereof of low weight-average molecular weights, quaternary polymers of vinylpyrrolidone and of vinylimidazole, optionally crosslinked homopolymers or copolymers of methacryloyloxy(C₁-C₄)alkyltri(C₁-C₄)alkylammonium salts, and mixtures thereof.

If they are present, the cationic polymers may be present in concentrations ranging from 0.01% to 20% by weight, preferably from 0.05% to 10% by weight and more particularly from 0.1% to 5% by weight, relative to the total weight of the composition.

The composition according to the invention is preferably aqueous. It may comprise water and optionally at least one organic solvent, chosen especially from C₁-C₄ alcohols such as ethanol, isopropanol, tert-butanol or n-butanol; polyols such as glycerol, propylene glycol and polyethylene glycols.

Preferably, the composition comprises from 70% to 95% by weight of water relative to the total weight of the composition.

The pH of the composition according to the invention is generally between 2 and 11, preferably between 3 and 10 and better still between 4 and 8.

The composition according to the invention may also comprise additives chosen from anionic polymers, nonionic polymers, amphoteric polymers, associative or non-associative polymeric thickeners, non-polymeric thickeners, nacreous agents, opacifiers, dyes, pigments, fragrances, vitamins, UV-screening agents, free-radical scavengers, antidandruff agents, preserving agents, pH stabilizers and cationic surfactants, and mixtures thereof. A person skilled in the art will take care to select the optional additives and the amount thereof such that they do not harm the properties of the compositions of the present invention.

These additives may be present in the composition according to the invention in an amount ranging from 0.01% to 20% by weight relative to the total weight of the composition.

The compositions according to the invention may be packaged in various forms, especially in bottles, in pump bottles or in aerosol containers so as to apply the composition in vaporized form or in the form of a mousse. The compositions may also impregnate applicators, especially gloves or wipes.

The composition according to the invention may be applied after dyeing, in particular oxidation dyeing, permanent-waving or relaxing the hair, or any other haircare treatment, in particular treatments using one or more cationic polymers.

In a preferred variant of the invention, the composition according to the invention may be applied after dyeing, in particular oxidation dyeing.

The present invention also relates to a cosmetic treatment process, especially for conditioning keratin materials, in particular human keratin fibers and most especially the hair, or even sensitized hair, in which a composition as defined above is applied to said keratin materials, and rinsing is optionally performed, after an optional leave-on time.

It is most particularly a process in which the application of the composition is performed after dyeing, in particular oxidation dyeing, permanent-waving or relaxing the hair, or any other haircare treatment, and preferably after dyeing, in particular oxidation dyeing.

The examples that follow are given as illustrations of the present invention. Unless otherwise mentioned, all the amounts indicated are expressed as weight percentages of commercial material.

### EXAMPLE 1

The following hair-conditioning composition was prepared:

COMPOSITION 1

| | |
|---|---|
| Cetylstearyl alcohol (50/50 C16/C18 by weight) | 7% |
| (Lanette O OR Flakes from Cognis) | |
| Myristyl/cetyl/stearyl myristate/palmitate/stearate | 1.5% |
| mixture (Crodamol MS-V from Croda) | |
| Sodium cetostearyl sulfate (50/50 C16/C18) | 3% AM |
| (Lanette E Granules from Cognis) | |
| Amino silicone at 20% by weight in polydimethyl- | 0.5% |
| siloxane 20 cSt (KF 8020 from Shin-Etsu) | |
| Citric acid qs | pH 5 |
| Preserving agents | 0.33% |
| Water | qs 100% |

AM: active material

### EXAMPLE 2

The following hair-conditioning composition was prepared:

COMPOSITION 2

| | |
|---|---|
| Cetylstearyl alcohol (50/50 C16/C18) | 7% |
| (Lanette O OR Flakes from Cognis) | |
| Myristyl/cetyl/stearyl myristate/palmitate/stearate | 1.5% |
| mixture (Crodamol MS-V from Croda) | |
| Sodium cetostearyl sulfate (50/50 C16/C18) | 3% AM |
| (Lanette E Granules from Cognis) | |
| Amino silicone at 20% by weight in polydimethyl- | 3.5% |
| siloxane 20 cSt (KF 8020 from Shin-Etsu) | |
| Citric acid qs | pH 5 |
| Preserving agent | 0.33% |
| Water | qs 100% |

### EXAMPLE 3

The following hair-conditioning composition was prepared:

COMPOSITION 3

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 1% |
| mixture (Crodamol MS-V from Croda) | |
| Sodium cetostearyl sulfate (50/50 C16/C18) | 1% AM |
| (Lanette E Granules from Cognis) | |
| Amino silicone at 20% by weight in polydimethyl- | 3.5% |
| siloxane 20 cSt (KF 8020 from Shin-Etsu) | |
| Citric acid qs | pH 5 |
| Preserving agent | 0.33% |
| Water | qs 100% |

### EXAMPLE 4

The following hair-conditioning composition was prepared:

COMPOSITION 4

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 2% |
| mixture (Crodamol MS-V from Croda) | |
| Sodium cetostearyl sulfate (50/50 C16/C18) | 5% AM |
| (Lanette E Granules from Cognis) | |
| Amino silicone at 20% by weight in polydimethyl- | 3.5% |
| siloxane 20 cSt (KF 8020 from Shin-Etsu) | |
| Citric acid qs | pH 5 |
| Preserving agent | 0.33% |
| Water | qs 100% |

### EXAMPLE 5

The following hair-conditioning composition was prepared:

COMPOSITION 5

| | |
|---|---|
| Cetylstearyl alcohol (50/50 C16/C18) | 5% |
| (Lanette O OR Flakes from Cognis) | |
| Myristyl/cetyl/stearyl myristate/palmitate/stearate | 1.5% |
| mixture (Crodamol MS-V from Croda) | |
| Sodium cetostearyl sulfate (50/50 C16/C18) | 1% AM |
| (Lanette E Granules from Cognis) | |
| Amino silicone at 20% by weight in polydimethyl- | 0.5% |
| siloxane 20 cSt (KF 8020 from Shin-Etsu) | |
| Citric acid qs | pH 5 |
| Preserving agent | 0.33% |
| Water | qs 100% |

### EXAMPLE 6

The following hair-conditioning composition was prepared:

COMPOSITION 6

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 1% |
| mixture (Crodamol MS-V from Croda) | |
| Sodium cetostearyl sulfate (50/50 C16/C18) | 1% AM |
| (Lanette E Granules from Cognis) | |
| Dimethicone (Dow Corning 200 Fluid 500000 cSt | 0.65% |
| from Dow Corning) | |
| Dimethicone (Mirasil DM 50 from Bluestar) | 1.95% |
| Citric acid qs | pH 5 |
| Preserving agent | 0.33% |
| Water | qs 100% |

### EXAMPLE 7

The following hair-conditioning composition was prepared:

COMPOSITION 7

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 2% |
| mixture (Crodamol MS-V from Croda) | |
| Sodium cetostearyl sulfate (50/50 C16/C18) | 5% AM |
| (Lanette E Granules from Cognis) | |
| Dimethicone (Dow Corning 200 Fluid 500000 cSt from | 0.65% |
| Dow Corning) | |
| Dimethicone (Mirasil DM 50 from Bluestar) | 1.95% |
| Citric acid qs | pH 5 |
| Preserving agent | 0.33% |
| Water | qs 100% |

### EXAMPLE 8

The following hair-conditioning composition was prepared:

COMPOSITION 8

| | |
|---|---|
| Cetylstearyl alcohol (50/50 C16/C18) | 5% |
| (Lanette O OR Flakes from Cognis) | |
| Myristyl/cetyl/stearyl myristate/palmitate/stearate | 1.5% |
| mixture (Crodamol MS-V from Croda) | |
| Sodium cetostearyl sulfate (50/50 C16/C18) | 1% AM |
| (Lanette E Granules from Cognis) | |
| Dimethicone (Dow Corning 200 Fluid 500000 cSt | 0.65% |
| from Dow Corning) | |
| Dimethicone (Mirasil DM 50 from Bluestar) | 1.95% |
| Citric acid qs | pH 5 |
| Preserving agent | 0.33% |
| Water | qs 100% |

### EXAMPLE 9

The following hair-conditioning composition was prepared:

COMPOSITION 9

| | |
|---|---|
| Cetylstearyl alcohol (50/50 C16/C18) | 5% |
| (Lanette O OR Flakes from Cognis) | |
| Myristyl/cetyl/stearyl myristate/palmitate/stearate | 1.5% |
| mixture (Crodamol MS-V from Croda) | |
| Sodium cetostearyl sulfate (50/50 C16/C18) | 1% AM |
| (Lanette E Granules from Cognis) | |
| Octyldodecanol | 2% |
| Citric acid qs | pH 5 |
| Preserving agent | 0.33% |
| Water | qs 100% |

The compositions of Examples 1 to 9 are stable. If the sodium cetostearyl sulfate is replaced with sodium lauryl sulfate in composition 1, the composition is unstable.

Wet and dried hair, treated with the compositions of Examples 1 to 9, have very good cosmetic properties, especially in terms of sleeking.

### EXAMPLE 10

The following hair-conditioning composition was prepared:

COMPOSITION 10

| | |
|---|---|
| Cetylstearyl alcohol (50/50 C16/C18 by weight) | 7% |
| (Lanette O OR Flakes from Cognis) | |
| Myristyl/cetyl/stearyl myristate/palmitate/stearate | 1.5% |
| mixture (Crodamol MS-V from Croda) | |
| Disodium stearoyl glutamate | 3% AM |
| (Amisoft HS 21P from Ajinomoto) | |
| Amino silicone at 20% by weight in polydimethyl- | 0.5% |
| siloxane 20 cSt (KF 8020 from Shin-Etsu) | |
| Citric acid qs | pH 5 |
| Preserving agents | 0.33% |
| Water | qs 100% |

AM: active material

### EXAMPLE 11

The following hair-conditioning composition was prepared:

COMPOSITION 11

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 2.1% |
| mixture (Crodamol MS-V from Croda) | |
| Disodium stearoyl glutamate | 1% AM |
| (Amisoft HS 21P from Ajinomoto) | |
| Polydimethylsiloxane 500000 cSt | 0.65% |
| (Dow Corning 200 Fluid from Dow Corning) | |
| Polydimethylsiloxane 50 cSt (Mirasil DM 50 from | 1.95% |
| Rhodia) | |
| Citric acid qs | pH 5 |
| Preserving agent | 0.33% |
| Water | qs 100% |

### EXAMPLE 12

The following hair-conditioning composition was prepared:

COMPOSITION 12

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 2.1% |
| mixture (Crodamol MS-V from Croda) | |
| Sodium palmitoyl sarcosinate | 1% AM |
| (Nikkol Sarcosinate PN from Nikko) | |
| Polydimethyl/methylaminopropylsiloxane at 20% in | 0.5% |
| polydimethylsiloxane 20 cSt (KF 8020 from Shin-Etsu) | |
| Citric acid qs | pH 5 |
| Preserving agent | 0.33% |
| Water | qs 100% |

### EXAMPLE 13

The following hair-conditioning composition was prepared:

COMPOSITION 13

| | |
|---|---|
| Cetylstearyl alcohol (50/50 C16/C18) | 7% |
| (Lanette O OR Flakes from Cognis) | |
| Myristyl/cetyl/stearyl myristate/palmitate/stearate | 0.5% |
| mixture (Crodamol MS-V from Croda) | |
| Sodium behenoyl lactylate (Pationic SBL from Rita) | 1% AM |
| Citric acid qs | pH 5 |
| Preserving agent | 0.33% |
| Water | qs 100% |

### EXAMPLE 14

The following hair-conditioning composition was prepared:

COMPOSITION 14

| | |
|---|---|
| Cetylstearyl alcohol (50/50 C16/C18) | 10% |
| (Lanette O OR Flakes from Cognis) | |
| Myristyl/cetyl/stearyl myristate/palmitate/stearate | 2.1% |
| mixture (Crodamol MS-V from Croda) | |
| Disodium stearoyl glutamate | 1% AM |
| (Amisoft HS 21P from Ajinomoto) | |
| Octyldodecanol | 2% |
| Citric acid qs | pH 5 |
| Preserving agent | 0.33% |
| Water | qs 100% |

Wet and dried hair, treated with the compositions of Examples 10 to 14, have very good cosmetic properties, especially in terms of sleeking.

1.-18. (canceled)

19. A non-coloring and non-oxidizing cosmetic composition comprising at least one anionic surfactant chosen from sulfate, sulfonate and carboxylic anionic surfactants, at least one solid fatty alcohol, and at least one solid fatty ester; wherein the at least one anionic surfactant comprises at least 50% by weight of anionic surfactants comprising fatty chains containing greater than or equal to 14 carbon atoms.

20. The composition according to claim 19, wherein the anionic surfactants are chosen from:
alkyl sulfates, alkyl ether sulfates, alkylamido ether sulfates, alkylaryl polyether sulfates, monoglyceride sulfates, alkylsulfonates, alkylamidesulfonates, alkylarylsulfonates α-olefin sulfonates, paraffin sulfonates, alkylsulfosuccinates, alkyl ether sulfosuccinates, alkylamide sulfosuccinates, and alkylsulfoacetates;
alkylsulfosuccinates, alkyl ether sulfosuccinates, alkylamide sulfosuccinates, acyl glycinates, acyl sarcosinates, and acyl glutamates,
alkyl monoesters of polyglycoside-polycarboxylic acids such as alkyl glucoside citrates, alkyl polyglycoside tartrates and alkyl polyglycoside sulfosuccinates, and alkylsulfosuccinamates,
acyllactylates;
alkyl-D-galactosideuronic acids and salts thereof, polyoxyalkylenated (C14-30)alkyl ether carboxylic acids, polyoxyalkylenated (C14-30)alkyl(C6-30)aryl ether carboxylic acids, polyoxyalkylenated (C14-30)alkylamido ether carboxylic acids and salts thereof; and
mixtures thereof.

21. The composition according to claim 20, wherein the anionic surfactants are chosen from:
C16-C18 alkyl sulfates and C16-C18 alkyl ether sulfates, optionally in the form of alkali metal, alkaline-earth metal, ammonium, amine or amino alcohol salts;
acyl glutamates, acyl sarcosinates, acyl lactylates, optionally in the form of alkali metal, alkaline-earth metal, ammonium, amine or amino alcohol salts; and
mixtures thereof.

22. The composition according to claim 19, wherein the at least one anionic surfactant is present in a total amount ranging from about 0.1% to about 20% by weight, relative to the total weight of the composition.

23. The composition according to claim 19, wherein the at least one solid fatty alcohol is of structure R—OH, wherein R is a linear alkyl group, optionally substituted with at least one hydroxyl group, comprising from 12 to 40 carbon atoms.

24. The composition according to claim 23, wherein the at least one solid fatty alcohol is chosen from cetyl alcohol, stearyl alcohol, cetylstearyl alcohol, behenyl alcohol, and mixtures thereof.

25. The composition according to claim 19, wherein the at least one solid fatty alcohols are present in an amount ranging from about 0.01% to about 30% by weight, relative to the total weight of the composition.

26. The composition according to claim 19, wherein the at least one solid fatty ester is chosen from esters of saturated or unsaturated monoalcohols comprising at least 10 carbon atoms.

27. The composition according to claim 19, wherein the at least one solid fatty ester is chosen from myristyl myristate, cetyl myristate, stearyl myristate, myristyl palmitate, cetyl palmitate, stearyl palmitate, myristyl stearate, cetyl stearate, stearyl stearate, and mixtures thereof.

28. The composition according to claim 19, wherein the at least one solid fatty ester is present in an amount ranging from about 0.1% to about 10% by weight, relative to the total weight of the composition.

29. The composition according to claim 19, further comprising at least one liquid fatty substance chosen from liquid fatty alcohols, liquid fatty esters, silicone oils, C₆-C₁₆ hydrocarbons, hydrocarbons containing more than 16 carbon atoms, non-silicone oils of animal origin, plant oils of triglyceride type, synthetic triglycerides, fluoro oils, and mixtures thereof.

30. The composition according to claim 29, wherein the at least one liquid fatty substance is chosen from liquid fatty alcohols and silicone oils.

31. The composition according to claim 29, wherein the at least one liquid fatty substance is present in an amount ranging from about 0.01% to about 20% by weight, relative to the total weight of the composition.

32. The composition according to claim 19, further comprising at least one nonionic surfactant and/or at least one amphoteric surfactant.

33. The composition according to claim 19, further comprising at least one cationic polymer.

34. The composition according to claim 19, further comprising water and optionally at least one organic solvent.

35. A cosmetic treatment process, comprising:
applying to keratin materials a composition comprising at least one anionic surfactant chosen from sulphate, sulfonate and carboxylic anionic surfactants, at least one solid fatty alcohol, and at least one solid fatty ester; wherein the at least one anionic surfactant comprises at least 50% by weight of anionic surfactants comprising fatty chains containing greater than or equal to 14 carbon atoms; and
optionally rinsing the keratin materials after an optional leave-on time.

36. The process according to claim 35, wherein the keratin materials include human keratin fibers and hair.

37. The process according to claim 35, wherein the cosmetic treatment comprises conditioning the keratin materials.

38. The process according to claim 35, wherein the composition is applied after dyeing, permanent-waving, and/or relaxing the keratin materials.
